(19) United States
(12) Patent Application Publication
(10) Pub. No.: US 2009/0118376 A1
(43) Pub. Date: May 7, 2009

(54) Memantine Protects Inflammation-Related Degeneration of Dopamine Neurons Through Inhibition of Over-Activated Microglia and Release of Neurotrophic Factors from Astroglia

(75) Inventors: Hung-Ming Wu, Tainan (TW); Jau-Shyong Hong, Durham, NC (US); Ru-Band Lu, Tainan (TW)

Correspondence Address: WPAT, PC;INTELLECTUAL PROPERTY ATTORNEYS, 2030 MAIN STREET, SUITE 1300, IRVINE, CA, 92614, US

(73) Assignee: Ru-Band Lu, Tainan (TW)

(21) Appl. No.: 11/934,520

(22) Filed: Nov. 2, 2007

(51) Int. Cl. A61K 31/13; A61P 25/00

(52) U.S. Cl. 514/662

(57) ABSTRACT

This invention discloses that memantine (MMT) protects dopamine (DA) neurons damage through its potent anti-inflammatory effect by inhibiting microglial over-activation and the protection on DA neuron is a dose-dependent response. This invention also discloses that NADPH oxidase plays a critical role of neuroprotection of MMT and MMT therapy for neurodegeneration diseases acts in part through an alternative novel mechanism by reducing microglia-associated inflammation. In addition, this invention reveals that MMT is neurotrophic to DA neurons through the release of neurotrophic factors from astroglia.

### FIELD OF THE INVENTION

This invention relates to methods for N-methyl-D-aspartate (NMDA) receptor antagonist (such as Memantine) protecting dopamine (DA) neurons damage through inhibition of over-activated microglia and release of neurotrophic factors from astroglia.

### BACKGROUND OF THE INVENTION

Neurodegenerative diseases such Alzheimer's and Parkinson's diseases have been extensively investigated in recent years. However, effective therapies are still limited. In pathological studies of Alzheimer's disease, the hallmark is beta amyloid accumulation (senile plaque) around with activated microglia and neuron loss; in biological studies, acetylcholine (Ach) concentration deficiency particularly is in forebrain and N-methyl-D-aspartate (NMDA) receptor hyperactive. Many drugs were designed to increase Ach concentration by inhibiting Ach-degradation enzyme; however this kind of treatment can't modify the disease course. Memantine (MMT) was developed to decrease the hyperactivity of NMDA receptors and has been proved to be an effective therapy for moderate and severe dementia. In clinic trial, MMT is demonstrated to be effective in the treatment of dementia. Lipton, et al. had demonstrated well that MMT is an uncompetitive NMDA receptor antagonist and recommended the neuroprotective effect of MMT resulted from its blockade of NMDA receptor. (Lipton S A. Paradigm shift in neuroprotection by NMDA receptor blockade: memantine and beyond. Nat Rev Drug Discov. 2006; 5:160-70.Review). Dogan, et al. showed MMT protected neuron damage against a large increase in the release of glutamate from ischemia reperfusion in spontaneously hypertensive rats (Dogan A, Eras M A, Rao V L and Dempsey R J. (1999). Protective effects of MMT against ischemia-reperfusion injury in spontaneously hypertensive rats Acta Neurochir (Wien): 141(10):1107-13.). Furthermore, glia including astrocytes and microglia play an important role in balancing of glutamate uptake and release to prevent excito-toxic neuron damage.

### BRIEF DESCRIPTION OF DRAWINGS

FIG. 1 shows effect of MMT on LPS-induced neurotoxicity of DA neurons. Rat midbrain mixed neuron-glia cultures were seeded in 24-well plates and treated or pretreated with vehicle and various concentrations of MMT for 30 minutes followed by 2.5 ng/ml LPS for 7 days. Degeneration of DA neurons was evaluated with the ┌³H┘ DA uptake assay (A) or immunostained with anti-TH antibody followed by quantification of the positively stained cells (B and C). Values are mean ±SEM of three independent experiments. *p<0.05, compared with LPS or control.

FIG. 2 shows effect of post-treatment with MMT on LPS-induced neurotoxicity. Rat midbrain mixed neuron-glia cultures were post-treated with MMT (10 μM) at indicated time points after LPS (2.5 ng/ml) administration. Seven days later, the effect of MMT on neurotoxicity was determined by ³H┘ DA uptake capacity assay. Data are percentage of control cultures, and are mean ±SEM of three independent experiments. *p<0.05, compared with LPS.

FIG. 3 shows lack of effect of MMT on MPP⁺-induced DA neurodegeneration in neuron-enriched cultures. Midbrain neuron-enriched cultures were pretreated for 30 minutes with indicated concentrations of MMT followed by 0.25 μM MPP⁺. Seven days later, DA uptake capacity assay was performed. Data are percentage of control cultures, and are mean ±SEM of three independent experiments. *p<0.05, compared with MPP⁺.

FIG. 4 shows effect of MMT on LPS-induced microglia activation and inflammatory mediator release in mesencephalic neuron-glia cultures. MMT inhibited LPS-induced microglia activation. Ventral mesencephalic neuron-glia cultures were pre-treated for 30 min with vehicle or 10 μM MMT prior to treatment for 24 hours with 2.5 ng/ml LPS. Spare OX-42-IR microglia was observed in the cultures with vehicle and MMT treatment. LPS treatment led to an increase OX-42-IR micorglia. Images presented are from one experiment and representative of at least three independent experiments.

FIG. 5 shows inhibitory effect of MMT on LPS-induced inflammatory mediator release in mesencephalic neuron-glia cultures. Effects of MMT on LPS-stimulated superoxide production in enriched microglia cultures were determined as described under Materials and Methods. Ventral mesencephalic neuron-glia cultures were pretreated for 30 min with vehicle or indicated concentrations of MMT prior to treatment with 10 ng/ml of LPS (A). Intracellular ROS were determined at 2 hours (B). TNF-α production was determined at 4 hours (C). Levels of nitrite (D) and PGE₂ in the supernatant were determined at 24 or 48 hours (E). Data are percentage of control cultures, and are mean ±SEM of three independent experiments. *p<0.05, compared with control or LPS.

FIG. 6 shows PHOX impact on MMT neuroprotection. PHOX⁺/⁺(EM-C57) and PHOX⁻/⁻(EM-Cybb) mouse enriched microglia cultures were pretreated with vehicle or MMT for 30 min, followed by LPS treatment. Four hours later, supernatant was taken and TNF-α concentration was measured. Values are mean ±SEM of three independent experiments. *p<0.05, compared with LPS.

FIG. 7 shows MMT is lack of effect for enhanced apoptosis of activated microglia induced by LPS. HAPI was seeded with 1*10⁴/well in 96-well plate. Twenty-four hours later, it was treated with vehicle, MMT (10 uM), and LPS 100 ng/ml for 48 hrs. After adding MTT, cell viability was measured (A) and morphology (B) was examined by contrast microscope. Values are mean ±SEM of three independent experiments. *p<0.05, compared with LPS.

FIG. 8 shows MMT induces dose-dependent surviving-promoting effects against spontaneous DA neurons death in rat primary midbrain neuron-glia cultures. Rat primary mesencephalic neuron-glia cultures seeded in a 24-well culture plate at density of 5×10⁵ per well were treated with indicated concentrations of MMT or its vehicle seven days after seeding. Seven days later, the viability of DA neurons was assessed by ┌³H┘ DA uptake assays (A), TH-IR neuron counts (B).

FIG. 9 shows neurotrophic effect of MMT is astrocyte-dependent. Astrocytes, not micrglia, contribute to the neurotrophic effect of MMT. Neuron-enriched cultures were treated with vehicle and 1-10 μM MMT (A); 10% (5×10⁴/well) of microglia were added back to neuron-enriched cultures and treated with 10 μM MMT (B); Depleted microglia cultures were treated with 10 μM MMT (C). ┌³H┘ DA uptake was assayed 7 days after treatment. Values are mean ±SEM of three independent experiments. *p<0.05, compared with corresponding vehicle-treated control cultures.

FIG. 10 shows MMT lacks effect of astrocytogenesis. MMT does not induce more proliferation of astrocyte, and micrglia compared with control in rat primary midbrain neuron-glia cultures. Rat primary mesencephalic neuron-glia cultures seeded in a 24-well culture plate at density of 5×10⁵ per well were treated with 10 μM MMT or its vehicle, and simultaneously with 1 μl Brdu seven days after seeding. 24 hours later, the cultures was fixed with 3.7% of PDF for GFAF, iba-1, and DAPI staining.

FIG. 11 shows astrocytes conditioned medium elicits robust neurotrophic and survival-promoting effects. Conditioned medium derived from rat primary astroglial cultures treated with vehicle or 10 μM MMT were harvested after 24 hours of incubation. Midbrain neuroglia cultures seeded in 24-well plates at a density of 5×10⁵ cells/well were treated with vehicle, MMT, ACM or ACM-MMT for 7 days. Neurotrophic effect was quantified by [³H] DA uptake assay. The data are expressed as mean ±s.e.m. of percentage of vehicle-treated control cultures from four to five independent experiments performed in triplicate; *P<0.05 compared with the vehicle-treated control cultures; †P<0.05 compared with the corresponding ACM-treated cultures.

FIG. 12 shows glutamate and aspartate concentrations of primary midbrain cultures. Primary neuron-glia cultures seeded in 24-well plates at a density of 5×10⁵ cells/well for 7 days. Then, the cultures were treated with vehicle, 10 μM MMT, LPS 5 ng/ml, and MMT 10 μM and LPS 5 ng/ml, and supernatants derived from the primary neuron-glia cultures at indicated time points. Glutamate concentration (A) and aspartate concentration were not obviously different between these four subjects. (B)

### SUMMARY OF THE INVENTION

The present invention provides a method of treating or preventing a disease caused by microglial over-activation-mediated dopamine (DA) neurons damage comprising administering a subject in need of such treatment or prevention a therapeutically effective amount of an N-methyl-D-aspartate (NMDA) receptor antagonist.

The present invention also provides a method of providing a neuroprotective effect comprising administering a subject an effective amount of a NMDA receptor antagonist.

### DETAILED DESCRIPTION OF THE INVENTION

In the present invention, it has been found that MMT has an effect on microglia activation to protect neuron damage; and that MMT has an alternative role on glia to inhibit chronic inflammation of brain and then modify the course of dementia disease. In the present invention, the role of glial cells in MMT-elicited neuroprotection on DA neurons against LPS-induced inflammation is demonstrated by using a series of different midbrain primary neuron/glia cell cultures.

This invention is the first report describing a novel glia-dependent anti-inflammatory mechanism underlying the neuroprotective effect of MMT. This invention shown that the neuroprotective effect of MMT against LPS-induced DA toxicity in mixed midbrain neuron/glia cultures is mediated through the inhibition of microglial over-activation by reducing the release of pro-inflammatory factors, such as reactive oxygen species, NO and PGE₂. Furthermore, this invention also shown MMT-treated astroglia-derived conditioned media exerted a significant neurotrophic effect on DA neurons in microglia-depleted neuron/glia cultures. It appears that MMT causes the release of neurotrophic factor(s) from astroglia, which in turn was responsible for the neurotrophic effect. These findings provide important alternative mechanisms for the explanation of MMT-elicited neuroprotection.

The prevailing view as to how MMT is neuroprotective and has beneficial effects for Alzheimer dementia patients has focused on the blockade of NMDA receptors (Lipton S A. Paradigm shift in neuroprotection by NMDA receptor blockade: memantine and beyond. Nat Rev Drug Discov. 2006; 5:160-70.). It is well-known that MMT is a low affinity antagonist for NMDA receptor, many reports demonstrated potent neuroprotection by MMT in excitotoxin (such as glutamate, NMDA or gp 120)-induced neurodegeneration in neuron cultures prepared from either rodent cortex or cerebellum (Weller M, Finiels-Marlier F, Paul S M. (1993) NMDA receptor-mediated glutamate toxicity of cultured cerebellar, cortical and mesencephalic neurons: neuroprotective properties of amantadine and memantine. Brain Res. 613:143-8.). One of the key reasons for the variation of the proposed anti-inflammation theory of this invention from the NMDA receptor-blockade mechanism is due to different model of cell cultures used.

In these excitotoxin-induced neurotoxicity models, MMT has been clearly shown to be potent neuroprotector through the inhibition of open channel of NMDA receptors. However, most of these in vitro studies on MMT mainly use neuron cultures, which devoid the opportunity to investigate the role of glial cells in the neuroprotective effect of this compound. This invention focuses on the role microglia on chronic inflammation-related neurodegeneration. One of the advantages of using mixed neuron cultures or microglia-depleted nueon/glia cultures is allowed to investigate the interaction between neurons and glial cells. In this inflammation in vitro model, this invention showed the major protective of MMT was mediated through the inhibitory effect on microglia.

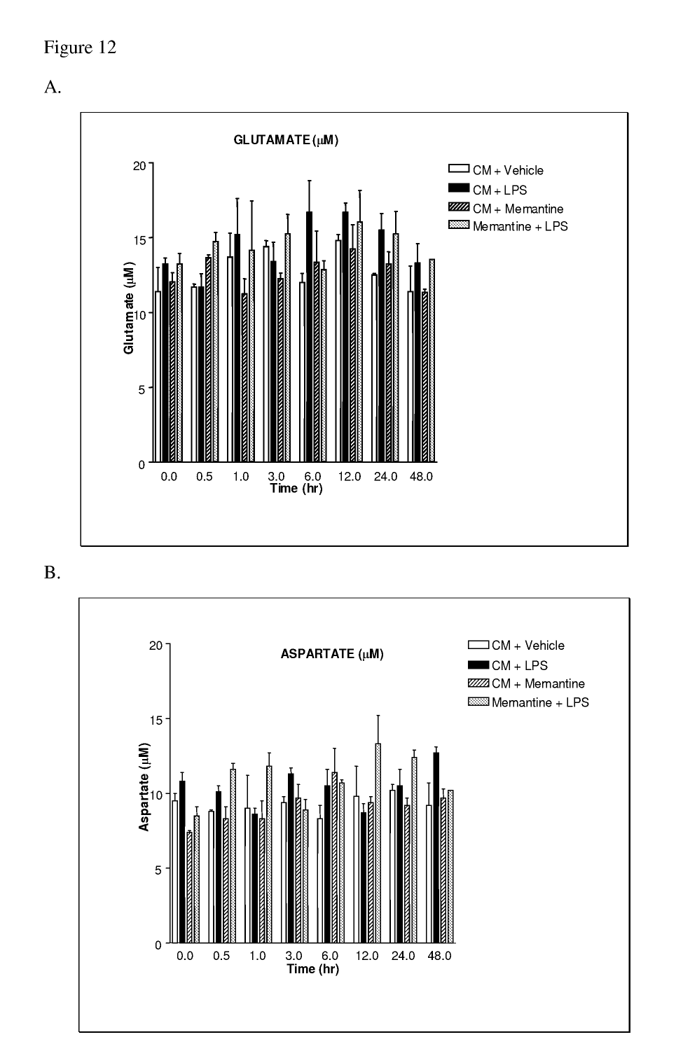

To determine the possibility that NMDA receptors might play a role in our mixed neuron/glia cultures in MMT-elicited neuroprotection, this invention determine the concentrations of excitatory amino acid, glutamate and aspartate in the supernatant of cultures after LPS treatment. Several authors reported the release of excitatory amino acid release from microglia by higher concentration of LPS (100 ng/ml), However, the concentration of glutamate released was limited to 10-20 μM, which may not be in sufficient concentrations to produce significant neuronal death (Obrenovitch T P, Urenjak J, Zilkha E, Jay T M. Excitotoxicity in neurological disorders—the glutamate paradox. Int J Dev Neurosci. 2000; 18:281-7.). In mixed neuron/glia cultures of this invention, with lower concentration of LPS (5 ng/ml) which was toxic to DA neurons, this invention could not detect any increases in both glutamate and aspartate (FIG. 12).

Again the difference can come from the difference in culture systems used. The previous report use enriched neuron cultures. However, in our neuro/glia cultures, even there was an increase in the release of glutamate, the level of this excitatory amino acid would remain low since it would be quickly taken up by astroglia.

The present invention demonstrated that NADPH oxidase, which is the key superoxide producing enzyme in microglia play a critical role in mediating the actions of MMT. Results from two sets of experiments support this conclusion. The first set of present invention used neuron/glia cultures prepared from NADPH oxidase-deficient mice (which lacks gp 91 subunit, and thus, unable to produce superoxide in the presence of LPS), MMT failed to produce any neuroprotective effect on LPS-induced neurotoxicity (preliminary data). The explanation came from our previous reports indicating that LPS causes release of pro-inflammatory factors from microglia by two pathways: a) to activation of CD14/TLR4 receptors to increase the gene expression of TNF-α and COX 2 and iNOS, and b) to stimulate the Mac1/NADPH oxidase pathway to increase the production of reactive oxygen species, which in turn would also increase the gene expression for some pro-inflammatory factors. Thus, the failure for MMT to protect LPS-induced DA neurons toxicity in NADPH oxidase-deficient neuron/glia cultures implies a critical role of this enzyme in mediating the neuroprotective effect of MMT. The present invention determines the binding site of MMT in microglia. Preliminary data using MMT to compete the binding of [³H]-labeled naloxine, which was shown in our laboratory to bind to gp 91, showed that MMT was effective in competing the binding a concentration manner (preliminary data). Since it was recently reported that no NMDA receptor was found in microglia by Wenk and his associates (Rosi S, Vazdarjanova A, Ramirez-Amaya V, Worley P F, Barnes C A, Wenk G L. (2006) Memantine protects against LPS-induced neuroinflammation, restores behaviorally-induced gene expression and spatial learning in the rat. Neuroscience. 142:1303-15.), the possibility for MMT binds to this receptor does not exist.

Accordingly, the present invention provides a method of treating or preventing a disease caused by microglial over-activation-mediated dopamine (DA) neurons damage comprising administering a subject in need of such treatment or prevention a therapeutically effective amount of N-methyl-D-aspartate (NMDA) receptor antagonist.

In the present, the treatment or prevention is made by inhibiting activation of microglial NADPH oxidase or by enhancing release of neurotrophic factor(s) from astroglia.

The term “NMDA receptor antagonist” as used herein is not limited but includes (i) a compound of formula I

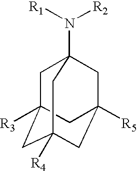

wherein
R₁, R₂, R₃, R₄ and R₅ are hydrogen or a straight or branched alkyl group of 1 to 6 C atoms; or a pharmaceutically-acceptable salt thereof;
(ii) (+)-5-methyl-10,11-dihydro-5H-dibenzo[a,d]cyclohepten-5,10-imine maleate (MK-801) or
(iii) 2-amino-5-phosphonopentanoate (AP-5).

In a preferred embodiment, the NMDA receptor antagonist is 1-amino-3,5-dimethyladamantane hydrochloride (MMT), MK-801 or AP-5.

In a more preferred embodiment, the NMDA receptor antagonist is 1-amino-3,5-dimethyladamantane hydrochloride (MMT).

In the present, the term “disease” is not limited but includes a neurodegenerative disease such as Parkinson's disease, Alzheimer's disease or dementia.

The term “subject” as used herein means any animal, such as a human, non-human primate, mouse, rat, guinea pig or rabbit.

The term “treating” as used herein means a subject afflicted with a disorder shall mean slowing, stopping or reversing the disorder's progression. In the preferred embodiment, treating a subject afflicted with a disorder means reversing the disorder's progression, ideally to the point of eliminating the disorder itself. In particular, treatment on the survival of dopamine neurons is a dose-dependent response.

In addition to neuroprotection against LPS-induced neurotoxicity, MMT is found to have high potency of neurotrophic effect on DA neurons in rat primary mesencephalic neuron-glia cultures. The neurotrophic effect of MMT was glia-dependent since MMT failed to show any protective effect in the neuron-enriched cultures. This invention subsequently demonstrated that it was the astroglia, not the microglia, which contributed to the neurotrophic effect of MMT. This conclusion was based on the reconstitution studies, in which we added 10% of microglia back to the neuron-enriched cultures or depleted microglia from neuron-glia culture, and found that MMT was neurotrophic in microlgia-depleted neuron/glia culture, but not microglia-added cultures.

Accordingly, the present invention provides a method of providing a neuroprotective effect comprising administering a subject an effective amount of a NMDA receptor antagonist.

### EXAMPLES

### Animals

Timed-pregnant (gestational day 14) adult female Fisher 344 rats were purchased from Charles River Laboratories (Raleigh, N.C., USA). Eight-wk-old (25-30 g) male and female B6.129S6-Cybb^{tm1Din} (PHOX^{-/-}) and C57BL/6J (PHOX^{+/+}) mice were purchased from Jackson Laboratories (Bar Harbor, Me., USA) and maintained in a strict pathogen free environment. The PHOX^{-/-} mice lack the functional catalytic subunit of the NADPH oxidase complex, gp91. NADPH oxidase is an inducible electron transport system in phagocytic cells that is responsible for the generation of the respiratory burst. PHOX^{-/-} mice are unable to generate extracellular superoxide in response to LPS or other immunological stimulus. Breeding of the mice was designed to achieve accurate timed-pregnancy ±0.5 days. Because the PHOX^{-/-} mutation is maintained in the C57BL/6J background, the C57BL/6J (PHOX^{+/+}) mice were used as control animals. Housing, breeding and experimental use of the animals were performed in strict accordance with the National Institutes of Health guidelines.

### Reagents

Lipopolysaccharide (LPS) (strain O111:B4) was purchased from Calbiochem (San Diego, Calif., USA). Cell culture ingredients were obtained from Life Technologies (Grand Island, N.Y., USA). [³H] Dopamine (DA, 28 Ci/mmol) and was purchased from NEN Life Science (Boston, Mass., USA). The polyclonal antibody against tyrosine hydroxylase (TH) was a kind gift from Dr. John Reinhard of Glaxo Wellcome (Research Triangle Park, N.C., USA). The neuron-specific nuclear protein (Neu-N) monoclonal antibody and the monoclonal antibody raised against the CR3 compliment receptor (OX42) were obtained from PharMingen (San Diego, Calif., USA). The biotinylated horse anti-mouse and goat anti-rabbit secondary antibodies were purchased from Vector Laboratories (Burlingame, Calif., USA). 2′,7′-Dichlorofluorescin diacetate (DCFH-DA) was obtained from Calbiochem (San Diego, Calif., USA). WST-1 was purchased from Dojindo Laboratories (Gaithersburg, Md., USA). TNF-α enzyme-linked immunosorbent assay (ELISA) kits were purchased from R&D Systems Inc. (Minneapolis, Minn., USA). PGE₂ ELISA kits were purchased from Cayman Chemical Company (Ann Arbor, Mich., USA). All other reagents came from Sigma Aldrich Chemical Co. (St. Louis, Mo., USA).

### Cell Samples

### Mesencephalic Neuron-Glia Cultures

Rat and mouse ventral mesencephalic neuron-glia cultures were prepared using a described protocol (Gao H M, Hong J S, Zhang W Q, Liu B (2002) Distinct Role for Microglia in Rotenone-Induced Degeneration of Dopaminergic Neurons. J Neurosci 22(3):782-790.). Briefly, midbrain tissues were dissected from day 14 Fisher 344 rat embryos or day 14 mouse embryos (PHOX^{+/+} or PHOX^{-/-}). Cells were dissociated via gentle mechanical trituration in minimum essential medium (MEM) and immediately seeded (5×10⁵/well) in poly D-lysine (20 μg/mL) precoated 24-well plates. Cells were seeded in maintenance media and treated with the treatment media described previously (Gao H M, Hong J S, Zhang W Q, Liu B (2002) Distinct Role for Microglia in Rotenone-Induced Degeneration of Dopaminergic Neurons. J Neurosci 22(3):782-790.). Three days after seeding, the cells were replenished with 500 μL of fresh maintenance media. Cultures were exposed 7 days after seeding. At the time of treatment, the composition of the cultures was approximately 48% astrocytes, 11% microglia, 40% neurons, and 1 to 1.5% TH-immunoreactive (ir) neurons.

### Neuron-Enriched Cultures

Mesencephalic neuron-glia cultures were seeded (5×10⁵/well) in 24 well plates precoated with poly D-lysine. Thirty-six hours postseeding, 5-10 μM cytosine β-D-arabinofuranoside was added to the culture. After 2 days, the cytosine β-D-arabinofuranoside was removed and replaced with fresh media. Neuron-enriched cultures are 98% pure, as indicated by ICC staining with OX-42 and GFAP antibodies. Neuron-enriched cultures were treated 7 days post-seeding. For microglia add-back cultures, the microglia were plated on top of the neuron-enriched culture at 6 days postseeding, resulting in the addition of either 10% (500 μL of 1×10⁵) or 20% (500 μL of 2×10⁵) microglia. Cells were treated 7 days after the initial seeding of the neuron-enriched cultures.

### Rat Astroglial Cultures

Mixed-glia cultures were first prepared from brains of 1-day-old Fisher 344 rat pups, as described previously. Briefly, mechanically dissociated brain cells (5×10⁷) were seeded onto 150-cm² culture flasks in Dulbecco's modified Eagle's medium containing 10% heat-inactivated FBS, 2 mM L-glutamine, 1 mM sodium pyruvate, 100 μM non-essential amino acids, 50 U/ml penicillin and 50 μg/ml streptomycin. The cultures were maintained at 37° C. in a humidified atmosphere of 5% CO₂ and 95% air, and medium was replenished 4 days after the initial seeding. Upon reaching confluence (usually 12-14 days later), microglia were detached from astrocytes by shaking the flasks at a speed of 180 r.p.m. for 5 h. Astrocytes were then detached with trypsin-ethylenediaminetetraacetic acid (EDTA) and seeded in the same culture medium. After five or more consecutive passages, cells were seeded onto 24-well plates (10⁵/well) for experiments. Immunocytochemical staining of the astroglial cultures with either anti-glial fibrillary acidic protein or anti-OX-42 antibody indicated an astrocyte purity of greater than 98% and less than 2% of microglia contamination.

### BV-2 Microglia Cell Line Cultures

The BV-2 cells were maintained in DMEM containing 10% heat-inactivated fetal bovine serum, 100 U/mL penicillin and 100 μg/mL streptomycin at 37° C. in a humidified incubator under 5% CO₂. Confluent cultures were passaged by E.D.T.A. trypsinization.

### Statistical Analysis

The data were expressed as the mean ±S.E.M. statistical significance was assessed with an analysis of variance followed by Bonferroni's t test using the Statview program (Abacus concepts, Berkeley, Calif.). A value of p<0.05 was considered statistically significant data are expressed as mean ±S.E.M.

### Example 1

### Uptake Assays and Cell Counting

### 1. [³H] DA Uptake Assays

Cells were incubated in Krebs-Ringer buffer (16 mM NaH₂PO₄, 1.2 mM MgSO₄, 1.3 mM EDTA, 4.7 nM KCL, for 21 min at 37° C. with 1 μM [³H] DA. Nonspecific uptake was blocked for DA with 10 μM mazindole. After incubation, cells were washed three times with 1 mL/well of ice-cold Krebs-Ringer buffer. Cells were then lysed with 0.5 mL/well of 1 N NaOH and mixed with 15 mL of scintillation fluid. Radioactivity was measured on a scintillation counter, where specific [³H] DA uptake was calculated by subtracting the mazindole.

### 2. Cell Counting

For visual counting of TH-ir neurons after Immunostaining, nine representative areas per well of the 24-well plate were counted under the microscope at 100¥ magnification. To measure the average TH-ir dendrite, 50 TH-ir representative neurons in each well were selected and three wells for each treatment condition were selected. In addition, the average dendrite length of TH-ir neurons was measured (Liu Y X, Qin L, Wilson B C, An L, Hong J S and Liu B (2002b) Inhibition by naloxone stereoisomers of -amyloid peptide (1-42)-induced superoxide production in microglia and degeneration of cortical and mesencephalic neurons. J Pharmacol Exp Ther 302: 1212-1219.).

### Results:

### Protective and Trophic Effects of MMT Alone and on LPS-Induced Degeneration of DA Neurons in Neuron/glia Cultures

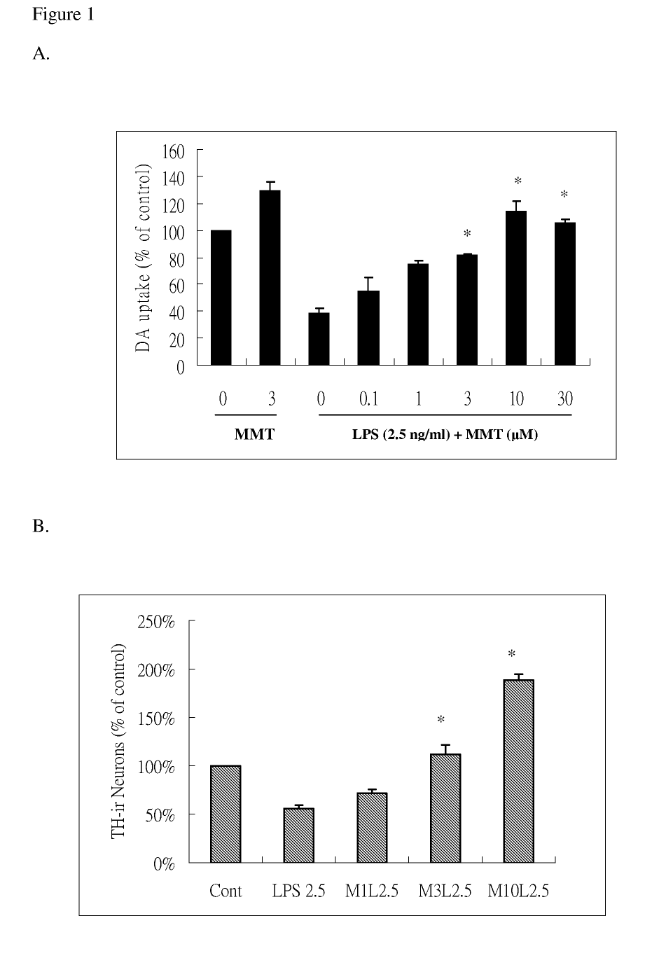

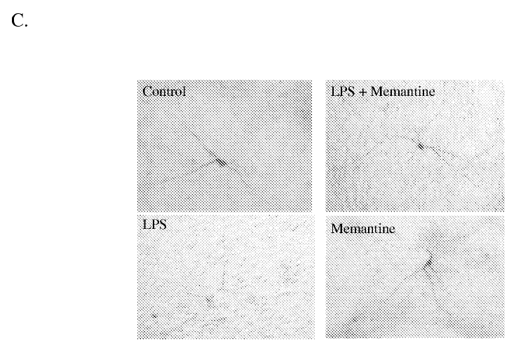

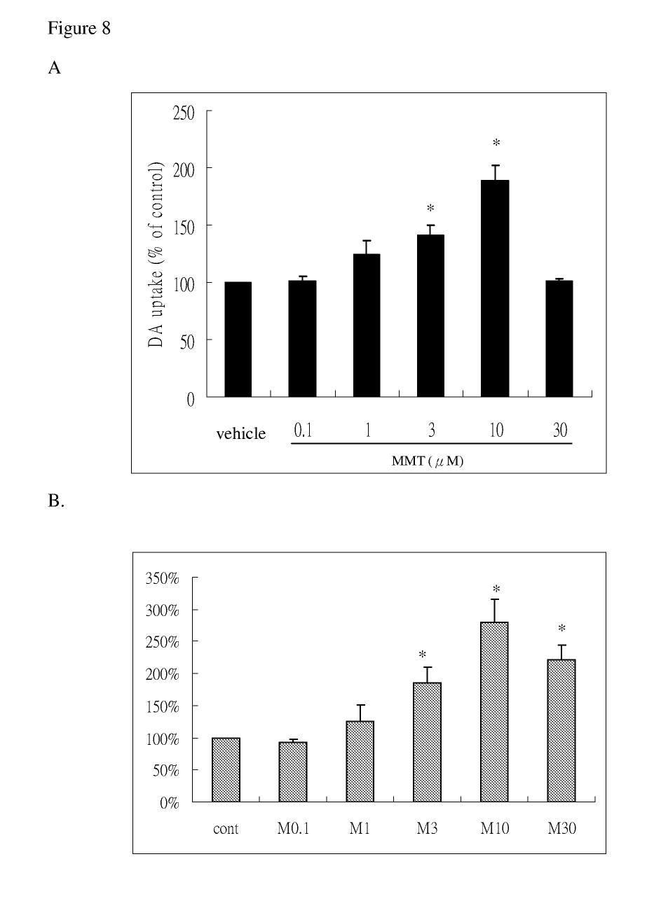

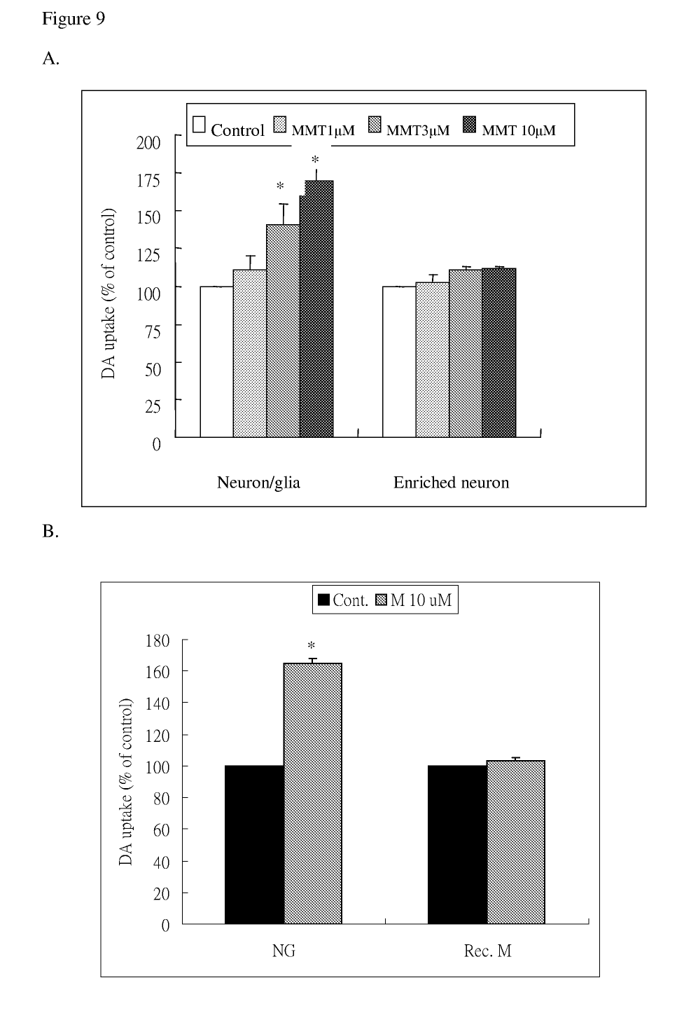

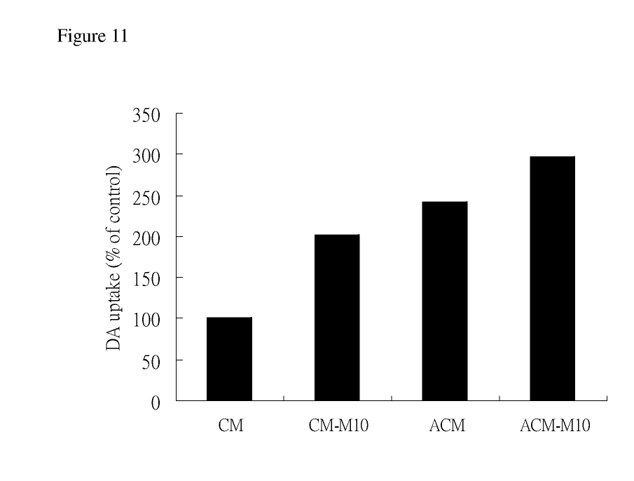

Rat mesencephalic neuron-glia cultures were pretreated for 30 min with vehicle or 1, 3, 10 μM MMT before adding LPS (2.5 to 5 ng/ml) to the cultures. One week later, the neurotoxic effect of LPS on DA neurons were assessed by both [H³] DA uptake, which measures the functional capacity of high affinity uptake of DA cells and cell count of tyrosine hydroxylase-positive (TH-ir) cells. [H³] DA uptake assays indicated that LPS treatment reduced uptake capacity to 42% of that vehicle-treated control cultures (FIG. 1A). MMT alone increased the uptake capacity by 30-80% in 3 and 10 μM of MMT, respectively. In addition, MMT significantly attenuated the LPS-induced decrease in DA uptake, in a dose-dependent manner (FIG. 1A), but not in neuron-enriched cultures (FIG. 1B). MMT alone can induce dose-dependent surviving-promoting effects against spontaneous DA neurons death in rat primary midbrain neuron-glia cultures (FIG. 8A). The neurotrophic effect of MMT is astrocyte-dependent. Astrocytes, not micrglia, contribute to the neurotrophic effect of MMT at 1-10 μM MMT (FIG. 9). Further the present invention also showed that astrocytes conditioned medium elicits robust neurotrophic and survival-promoting effects. (FIG. 11)

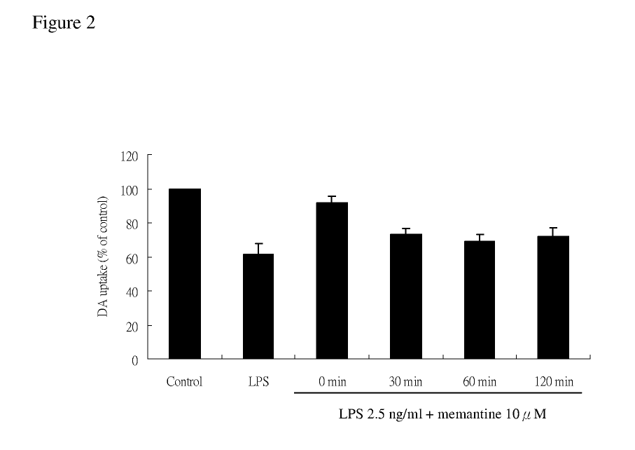

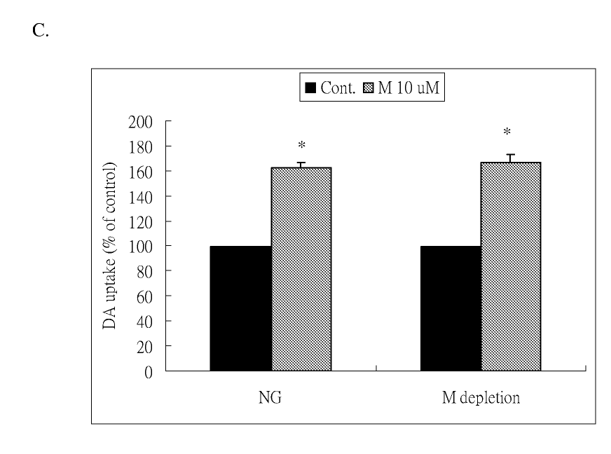

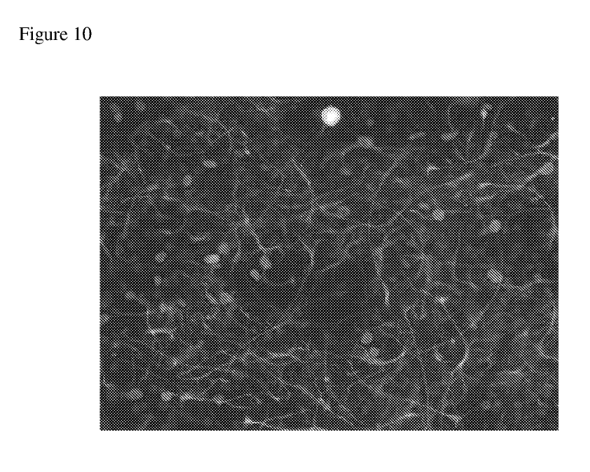

Parallel to the finding of DA uptake studies, analysis of cell count of the number of TH-ir neurons revealed that MMT alone increased the survival DA neuron number compared with vehicle control group (FIG. 1C and FIG. 8B). Morphological observation showed that MMT not only increased the number of DA neurons, but also enhanced the growth of neurites (FIG. 1D). Moreover, MMT (3 and 10 μM) significantly attenuated the LPS-induced reduction in the number of TH-ir neurons (FIG. 1C). In addition to the pre-treatment experiments, similar studies using post-treatment designs were conducted to determine the efficacy of MMT. In these experiments, neuron-glia cultures were either treated with MMT (10 μM) and LPS (2.5 ng/ml) at the same time, or MMT was added 30, 60, 120 or 180 min after the addition of LPS. One week later, DA uptake of the culture was assayed. Significant neuroprotection was observed in cultures in cultures with MMT added up to 120 min after the addition of LPS (FIG. 2). In cytogenesis test, MMT does not induce more proliferation of astrocyte, and micrglia compared with control in rat primary midbrain neuron-glia cultures, that MMT is lack of effect of astrocytognesis in neuron-glia culture (FIG. 10).

### Lack of Neuroprotective Effect of MMT in MPP⁺-Induced Neurotoxicity in Neuron-Enriched Cultures

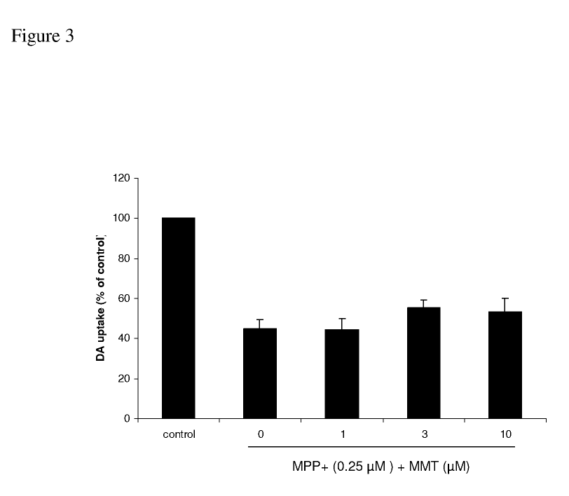

To determine whether the neuroprotective effect of MMT was dependent on the presence of glial cells, the effects of MMT on the neuron-enriched cultures were determined. The cultures contained 95% neurons and up to 5% astroglia (50% astroglia in normal neuron/glia cultures), after treatment with MPP⁺. Seven days after the treatment of MPP⁺, DA uptake was reduced by 31% compared with the control cultures. Pre-treatment of the neuron-enriched cultures with MMT (1, 3, or 10 μM) failed to protect MPP⁺-induced reduction in DA uptake (FIG. 3). These results suggested that the neuroprotective effect of MMT was dependent of the presence of glial cells.

### Lack of Neuroprotective Effect of MMT in LPS-Induced Neurotoxicity in Microglia-Depleted Neuron-/Glia Cultures

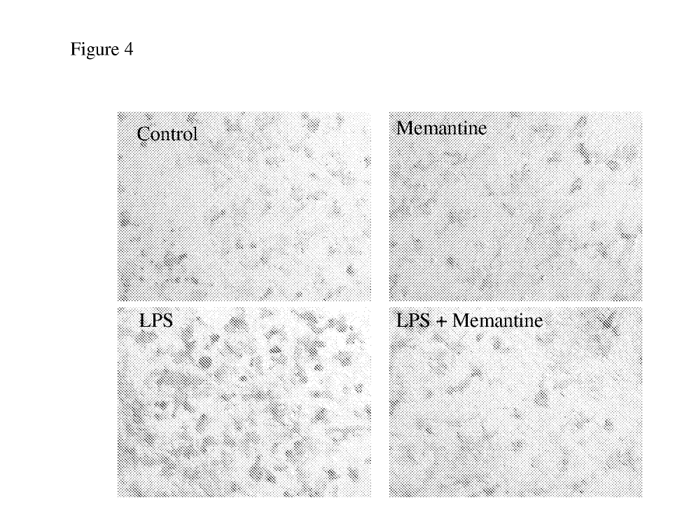

To evaluate the influence of various kinds of glia contribute to effect of MMT on LPS-induced dopaminergic neurotoxicity, microglia-depleted Neuron-/glia Cultures were performed. The data shown in FIG. 4 indicate that a protective effort was observed in the presence of microglia, but not found in depletion of microglia in neuron-glia mixed cultures by LME, which decreased microglial component to <1% of total cells in the mixed cultures microlgia-depletion cultures treated with LPS for 7 days. It is suggested that microglia contributed to neuroprotection against LPS-induced dopaminergic neurotoxicity.

### Example 2

### Immunostaining, Superoxide, Intracellular Reactive Oxygen Species, TNF-α, PGE₂ and Nitrite Assay

### 1. Immunostaining

DA neurons were recognized with the polyclonal antibody against tyrosine hydroxylase (TH) and microglia was detected with the OX-42 antibody against CR3 receptor. Briefly, cells were fixed for 20 min at room temperature in 3.7% formaldehyde diluted in phosphate-buffered saline (PBS). After washing twice with PBS, the cultures were treated with 1% hydrogen peroxide for 10 min. The cultures were again washed three times with PBS, then incubated for 40 min with blocking solution (PBS containing 1% bovine serum albumin (BSA), 0.4% Triton X-100, and 4% goat serum. The cultures were incubated overnight at 4° C. with the primary antibody diluted in DAKO antibody diluent and the cells were washed three times for 10 min each in PBS. The cultures were next incubated for 1 h with PBS containing 0.3% Triton X-100 and the appropriate biotinylated goat anti-rabbit secondary antibody (1:227). After washing three times with PBS, the cultures were incubated for 1 h with the Vectastain ABC reagents diluted in PBS containing 0.3% Triton X-100. Cells were then washed twice with PBS; the bound complex was visualized by incubating cultures with 3,3′-diaminobenzidine. Color development was halted by removing the reagents and washing the cultures twice with fresh PBS. To quantify cell numbers, nine representative areas per well in the 24-well plate were counted under the microscope at 100× magnification by two individuals. The average of these scores was reported.

### 2. Superoxide Assay

Extracellular superoxide (O₂⁻) production from microglia was determined by measuring the superoxide dismutase (SOD) inhibitable reduction of 2-(4-Iodophenyl)-3-(4-nitrophenyl)-5-(2,4-disulfophenyl)-2H-tetrazolium, monosodium salt, WST-1. Briefly, 200 μL of primary enriched-microglia were seeded (1×10⁵/well) in 96-well plates. The cells were then incubated for 24 h at 37° C. in a humidified atmosphere of 5% CO₂ and 95% air. Immediately before treatment, cells were washed twice with Hanks balanced salt solution (HBSS). To each well, 100 μL of HBSS with or without SOD (600 U/mL), 50 μL of vehicle or LPS, and 50 μL of WST-1 (1 mM) in HBSS were added. The cultures were incubated for 30 min at 37° C. and 5% CO₂ and 95% air. The absorbance at 450 nm was read with a Spectra Max Plus microtiter plate spectrophotometer (Molecular Devices, Sunnyvale, Calif., USA). Cell free experiments with and without substance P were conducted to determine that SP did not alter absorbance by itself. The amount of SOD-inhibitable superoxide was calculated and expressed as percent of vehicle-treated control cultures.

### 3. Intracellular Reactive Oxygen Species Assay

The production of intracellular reactive oxygen species (ROS) was measured by DCFH oxidation. The DCFH-DA reagent passively enters cell where it is de-acetylated by esterase to nonfluorescent DCFH. Inside the cell, DCFH reacts with ROS to form DCF, the fluorescent product. For this assay, 10 mM DCFH-DA was dissolved in methanol and was diluted 500-fold in HBSS to give a 20 μM concentration of DCFH-DA. Enriched-microglia cultures seeded (5×10⁴) in 96-well plates were then exposed to DCFH-DA for 1 h, followed by treatment with HBSS containing several concentrations of LPS or substance P for 2 h. After incubation, the fluorescence was read at the 485 nm excitation and 530 nm emission on a fluorescence plate reader. Cell free experiments with and without SP were conducted to determine that SP did not alter fluorescence by itself. To calculate the amount of intracellular ROS produced, the mean control treatment was subtracted from the mean treatment group.

### 4. TNF-α and PGE₂ Assay

The production of TNF-α was measured with a commercial ELISA kit from R&D Systems. The PGE₂ release was measured with a commercial ELISA kit from Cayman Chemical Company.

### 5. Nitrite Assay

As an indicator of nitric oxide production, the amount of nitrite accumulated in culture supernatant was determined with a calorimetric assay using Griess reagent [1% sulfanilamide, 2.5% H₃PO₄, 0.1% N-(1-naphthyl)ethylenediamine dihydrochloride]. Briefly, 50 μL of Griess reagent and 50 μL of culture supernatant were incubated in the dark at room temperature for 10 min. After incubation, the absorbance at 540 nm was determined with the Spectra Max Plus microplate spectrophotometer. The sample nitrite concentration was determined from a sodium nitrite standard curve.

### Results:

### Inhibition by MMT of LPS-induced Microglial Activation and Release of Pro-inflammatory Factors

To provide evidence of anti-inflammatory effect of MMT, the degree of inhibition of LPS-induced activation of microglia was determined by 1) morphological observation after immunostaining of microglia marker (OX-42) and 2) release of pro-inflammatory factors from activated microglia, such as extracelloualr superoxide radicals, intracellular reactive oxygen species (iROS), nitric oxide (NO), PGE₂.

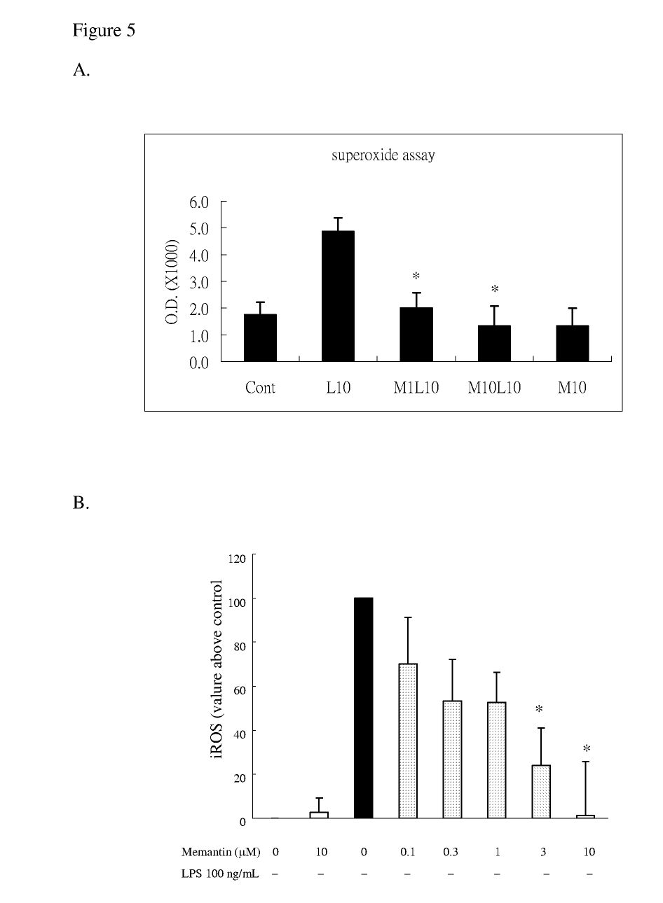

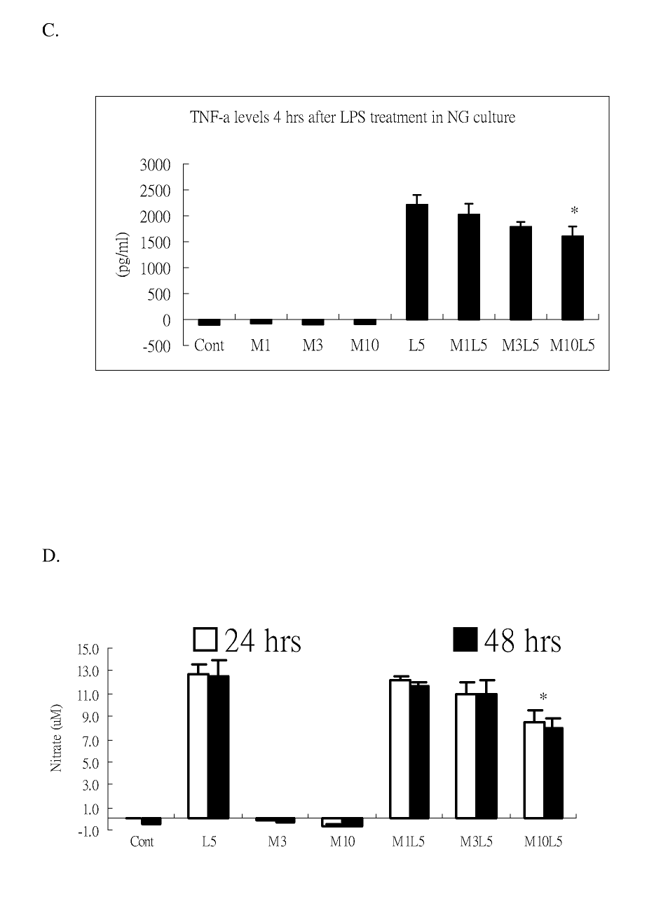

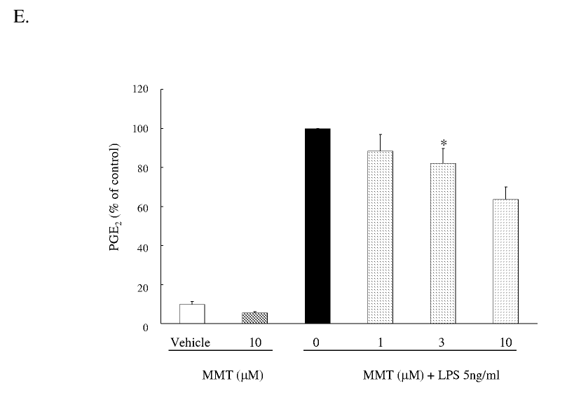

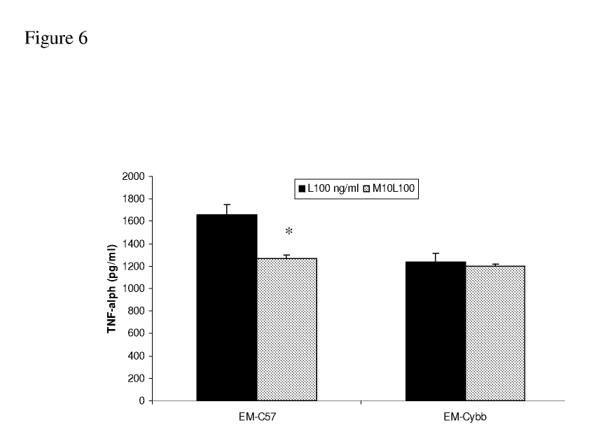

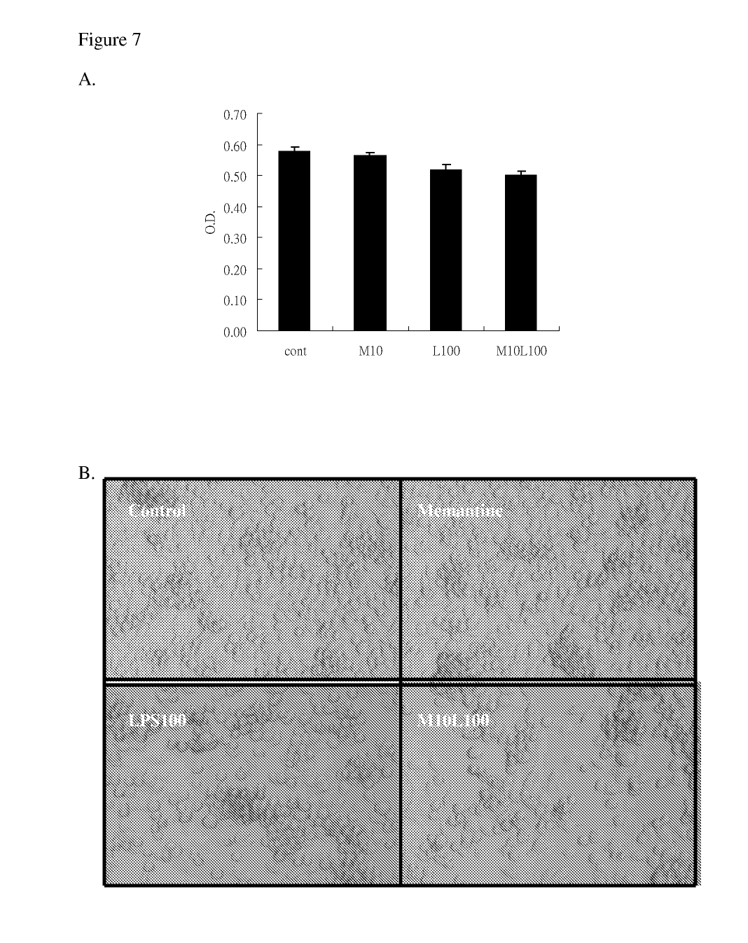

Neuron-glia cultures were pretreated for 30 min with MMT (3 μM) or vehicle before LPS stimulation. Twelve hours after LPS treatment, OX-42 stained microglia cells in the cultures pretreated with MMT were less activated than that of the LPS-treated cultures (FIG. 5). Production of superoxide (30 min after LPS) and iROS (2 h after LPS) was decreased by MMT treatment (FIGS. 5 A and B). In addition the release of TNF-α (4 h after LPS treatment) and NO (measured as nitrite) (24 and 48 h after LPS stimulation) was also reduced in MMT-treated samples (FIGS. 5 C and D). The production of PGE₂ in cultures pretreated with 3 and 10 μM MMT decreased by 23% and 27% respectively (FIG. 5E).

What is claimed is:

1. A method of treating a disease caused by microglial over-activation-mediated dopamine (DA) neurons damage comprising administering a subject in need of such treatment a therapeutically effective amount of N-methyl-D-aspartate (NMDA) receptor antagonist.

2. The method according to claim 1, wherein treating is made by inhibiting activation of microglial NADPH oxidase.

3. The method according to claim 1, wherein treating is made by the enhancement of release of neurotrophic factor(s) from astroglia.

4. The method according to claim 1, wherein the NMDA receptor antagonist is (i) a compound of formula I 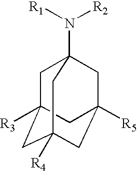
wherein
R₁, R₂, R₃, R₄ and R₅ are hydrogen or a straight or branched alkyl group of 1 to 6 C atoms; or a pharmaceutically-acceptable salt thereof;
(ii) (+)-5-methyl-10,11-dihydro-5H-dibenzo [a,d]cyclohepten-5,10-imine maleate (MK-801) or
(iii) 2-amino-5-phosphonopentanoate (AP-5).

5. The method according to claim 4, wherein the NMDA receptor antagonist is 1-amino-3,5-dimethyladamantane hydrochloride.

6. The method according to claim 1, wherein the disease is neurodegenerative disease.

7. The method according to claim 6, wherein the neurodegenerative disease is Parkinson's disease, Alzheimer's disease or dementia.

8. The method according to claim 1, wherein the subject is human.

9. (canceled)

10. A method of providing a neuroprotective effect comprising administering a subject an effective amount of a NMDA receptor antagonist.

11. The method according to claim 10, wherein the neuroprotective effect is made by inhibiting activation of microglial NADPH oxidase.

12. The method according to claim 10, wherein neuroprotection is made by the enhancement of release of neurotrophic factor(s) from astroglia.

13. The method according to claim 10, wherein the NMDA receptor antagonist is (i) a compound of formula I 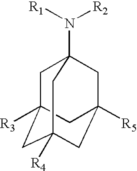
wherein
R₁, R₂, R₃, R₄ and R₅ are hydrogen or a straight or branched alkyl group of 1 to 6 C atoms; or a pharmaceutically-acceptable salt thereof;
(ii) (+)-5-methyl-10,11-dihydro-5H-dibenzo[a,d]cyclohepten-5,10-imine maleate (MK-801) or
(iii) 2-amino-5-phosphonopentanoate (AP-5).

14. The method according to claim 13, wherein the NMDA receptor antagonist is 1-amino-3,5-dimethyladamantane hydrochloride.

15. The method according to claim 10, wherein the subject is human.

16. (canceled)
